(19) United States
(12) Patent Application Publication
(10) Pub. No.: US 2008/0269710 A1
(43) Pub. Date: Oct. 30, 2008

(54) Hygiene article having a wipe and powdered substrate combination

(75) Inventors: David Joseph Caracci, Cincinnati, OH (US); Debra Lynn Hartsell, Cincinnati, OH (US); Sheri Dean Keeler, West Harrison, IN (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company

(21) Appl. No.: 11/796,212

(22) Filed: Apr. 27, 2007

(51) Int. Cl. A61F 13/84

(52) U.S. Cl. 604/385.06

(57) ABSTRACT

A packet, or packet combination, containing a wipe and powdered substrate is provided. The packet, or packet combination, may be used in conjunction with a hygiene article such as a sanitary napkin. The wipe is used to clean an area of skin or hair that has residual menstrual, urinary, or fecal material left behind after the removal of a hygiene article. The powder may then be used to provide an added benefit such as odor control. An order of use can be dictated by locating the packets containing the wipe and powdered substrate in specific positions on the hygiene article.

### FIELD OF THE INVENTION

The present invention relates generally to a wipe and powdered substrate combination, and more particularly to wipe and powdered substrate combinations used with hygiene articles.

### BACKGROUND OF THE INVENTION

Hygiene articles, such as incontinence devices, tampons, sanitary napkins, and diapers do not completely prevent body exudates from soiling areas of the skin. After the removal of a hygiene article, such areas may have residual menstrual, urinary, or fecal material attached to the skin or hair. It is also desirable to remove such residual exudate material from the body. Often, an individual who is removing a hygiene article is constrained to a rather limited geographical area such as a public restroom or a child's changing table. Therefore, unless the individual has some means to clean himself or herself within close proximity, they will remain soiled with the unwanted material.

In addition to the cleanliness concerns associated with the use of hygiene articles, there is often the problem of immediate and residual malodor. The malodor may occur during use, but is especially noticeable upon the removal of a hygiene article. Hygiene articles are designed to absorb materials such as menstrual fluid, urine, and feces, which have noticeable and unpleasant odors. These unpleasant odors, combined with the close contact of the hygiene article with the body surface, along with the general moistness of the areas where hygiene articles are worn, and the usually extended time period during which the hygiene article is worn, often while soiled, can cause an even greater malodor. The production and then release of this malodor during removal of a hygiene article is often distressing to the user.

Wipes have been used to clean the body after removal of hygiene articles. The user of a hygiene article will use a wipe to clean the area of skin associated with the wearing of the hygiene article. For example, when changing a sanitary napkin, the user may wish to wipe the vaginal area with a wet or dry wipe such as a disposable towelette. To provide users with ready access to wipes, prewrapped wipes have been provided with individual hygiene articles such as sanitary napkins. The prewrapped wipes have either been packaged with the hygiene article, or fixedly attached thereto.

Hygiene articles do not completely eliminate a “moist” feeling during use, particularly in an occluded area, or when the user is hot or sweaty. As such, it would be desirable to provide a product that would provide a clean dry feeling during or after use of a hygiene article. Further, it would be desirable to provide a wipe combination that could be modified according to specific uses. Additionally, it would be desirable to provide a product for use in combination with a hygiene article that would provide a pleasing smell to the user and increased odor control.

### SUMMARY OF THE INVENTION

A hygiene article is provided which comprises a hygiene article and a packet combination joined to the hygiene article. The packet combination includes a first packet comprising packaging material. The packaging material defines an outer surface of the packet and an internal space, wherein the internal space is a discrete compartment. The packet combination also includes a second packet comprising packaging material. The packaging material defines an outer surface of the packet and an internal space, wherein the internal space is a discrete compartment. The first packet contains at least one wipe disposed within the discrete compartment, and the second packet contains at least one powdered substrate disposed within the discrete compartment. The first packet and second packet are joined.

Another hygiene article is provided which comprises a hygiene article and a packet joined to the hygiene article. The packet comprises packaging material, wherein the packaging material defines an outer surface and an internal space. An internal divider is also present and is disposed within the internal space and joined to the packaging material to define a first discrete compartment and a second discrete compartment. The first discrete compartment contains at least one wipe and the second discrete compartment contains at least one powdered substrate.

### BRIEF DESCRIPTION OF THE DRAWINGS

While the specification concludes with claims particularly pointing out, and distinctly claiming the subject matter that is regarded as forming the present invention, it is believed that the invention will be better understood from the following description taken in conjunction with the accompanying Figures, in which:

FIG. 1 is a perspective view of a packet of the present invention.

FIG. 2 is a cross-sectional view of section A-A of FIG. 1 showing a packet comprising three packaging materials.

FIG. 3 is a cross-sectional view of section A-A of FIG. 1 showing a packet comprising two packaging materials.

FIG. 4 is a cross-sectional view of section A-A of FIG. 1 showing a packet comprising a single packaging material.

FIG. 5 is a perspective view of a packet combination of the present invention.

FIG. 6 is a cross-sectional view of section B-B of FIG. 5 showing a packet combination comprising two packaging materials.

FIG. 7 is a cross-sectional view of section B-B of FIG. 5 showing a packet combination comprising a single packaging material.

FIG. 8 is a perspective view of a packet combination of the present invention.

FIG. 9 is a cross-sectional view of section C-C of FIG. 8.

FIG. 10 is a partial cut-away perspective view of a powdered substrate of the present invention.

FIG. 11 is a perspective view of a powdered substrate and wipe.

FIG. 12 is a partial cut-away plan view of a sanitary napkin of the present invention.

FIG. 13 is a cross-sectional view of section D-D of FIG. 12 showing a packet and sanitary napkin.

FIG. 14 is a cross-sectional view of section D-D of FIG. 12 showing packets and sanitary napkin.

FIG. 15 is a plan view of a sanitary napkin of the present invention.

FIG. 16 is a cross-sectional view of section E-E of FIG. 15 showing a packet and sanitary napkin.

FIG. 17 is a plan view of a sanitary napkin of the present invention.

FIG. 18 is a cross-sectional view of section F-F of FIG. 17 showing a packet and sanitary napkin.

FIG. 19 is a perspective view of a sanitary napkin and packet of the present invention.

### DETAILED DESCRIPTION OF THE INVENTION

As used herein, “hygiene article” refers to articles that absorb, block, and/or contain body exudates discharged from the body, and which may be placed within, against or in proximity to the body of a user. Examples of hygiene articles include feminine hygiene pads, tampons, pessaries, inter labial pads, adult incontinence products, training pants, and baby diapers.

The term “joined”, as used herein, encompasses configurations in which a first element is directly secured to a second element. Joined also includes configurations in which the first element is indirectly secured to the second element by securing the first element to at least one intermediate member, which in turn is secured to the second element. Additionally, the term joined covers configurations in which the first element is integral with the second element, such that the first element is part of the second element. The first element and the second element can be fixedly joined, or releasably joined.

As used herein, the term “fixedly joined” refers to a configuration as defined under the term “joined”, where a first element cannot be separated from a second element without at least partially destroying one of the joined elements. As used herein, the term “releasably joined” also refers to a configuration as defined under the term “joined”, where a first element may be separated from a second element without causing destruction or undue distortion to either element.

Embodiments of the present invention are illustrated using the following figures. The present invention, however, is not limited to structures having the particular configurations shown in the figures.

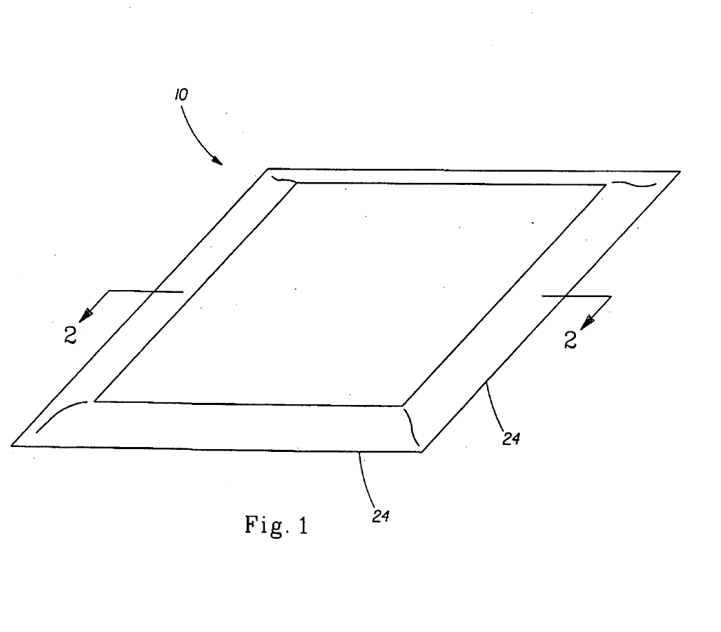

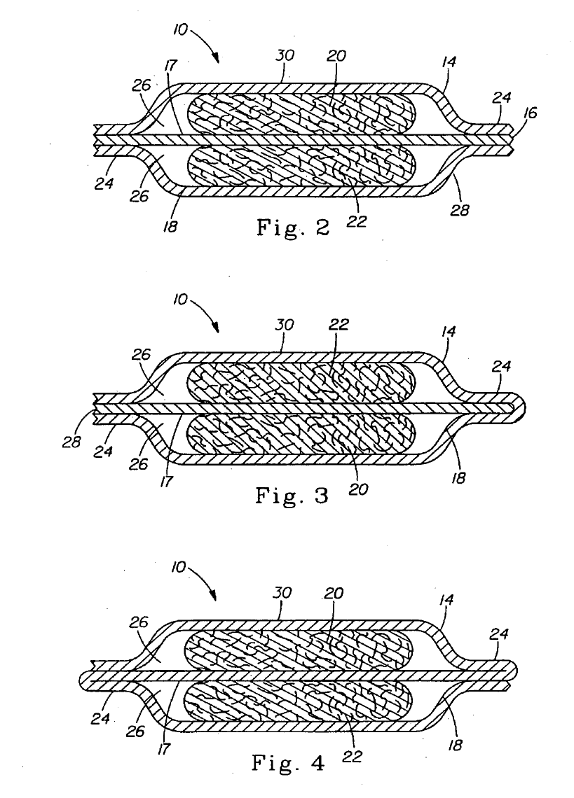

As shown in FIG. 1, in certain embodiments the present invention may comprise an individual packet 10. As used herein the term “individual packet”, or “packet”, refers to a structure comprising one or more packaging materials. The packaging materials form one or more discrete compartments. Further, a packet may be separate from other packets, or joined to other packets. A packet 10, as illustrated in FIGS. 2-4, which are cross-sections (A-A) of the packet 10 of FIG. 1, have two discrete compartments 26 each having a wipe 20 or powdered substrate 22 disposed therein. Each packet 10 contains at least one wipe 20 and/or at least one powdered substrate 22. While FIGS. 2-4 show the wipe 20 and powdered substrate 22 in specific discrete compartments 26 sharing a common internal divider 17, it is to be understood that the placement of the wipe 20 and powdered substrate 22 in the figures is for the purpose of illustration only. In certain embodiments, the packet 10 may have more than two discrete compartments 26, with each having a wipe 20 or powdered substrate 22 disposed therein. The discrete compartments 26 contain the wipe 20 and powdered substrate 22 in a single packet 10, and prevent the wipe 20 and powdered substrate 22 from contacting each other. In one embodiment, as shown in FIG. 2, a packet 10 and the discrete compartments 26 contained therein may be formed from three packaging materials. A first packaging material 14 and a second packaging material 28, forming at least a part of the outer surface 30 and at least a part of the inner surface 18 of the packet 10, and a third packaging material 16 forming an internal divider 17 disposed between the first packaging material 14 and the second packaging material 28.

In another embodiment, as shown in FIG. 3, a packet 10 and the discrete compartments 26 contained therein may be formed from two packaging materials. The first packaging material 14, forming at least a part of the outer surface 30 and at least a part of the inner surface 18 of the packet 10, and a second packaging material 28 forming an internal divider 17 in that the second packaging material 28 is disposed between the opposing inner surfaces 18 formed by the first packaging material 14. In another embodiment, as shown in FIG. 4, a packet 10 of the present invention may be formed from a single packaging material. The packet 10 and the discrete compartments 26 contained therein may be formed from a single packaging material, such as a first packaging material 14, which forms the outer surface 30, inner surface 18, and internal divider 17 of the packet 10. As shown in FIGS. 1-4, the packaging materials, which includes the first packaging material 14, second packaging material 28, and third packaging material 16, may have similar dimensions. The packet 10 may be formed by any method known to one of ordinary skill in the art, such as by joining the packaging materials about their respective peripheral edges 24 using heat and/or pressure seals, ultrasonic seals, glue seals, and zippered track sealing systems, such as Dow Company's “ZIP LOCKS”. In certain embodiments, the packet 10 may be hermetically sealed.

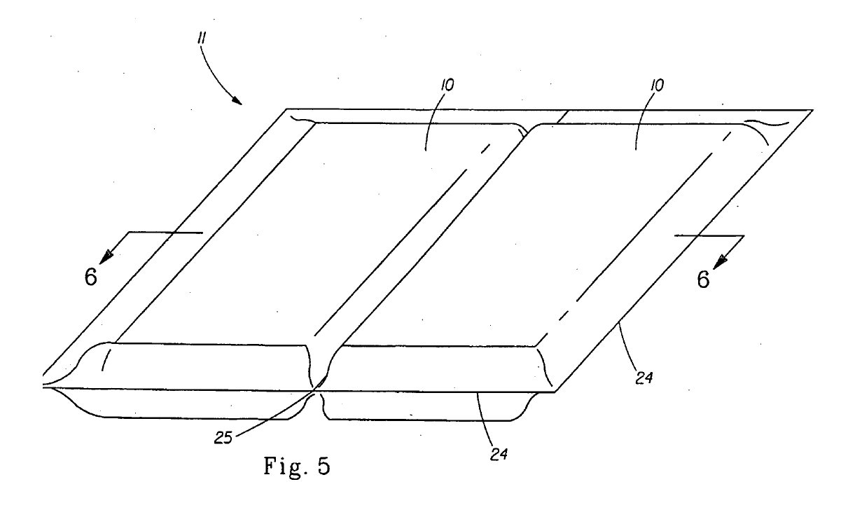

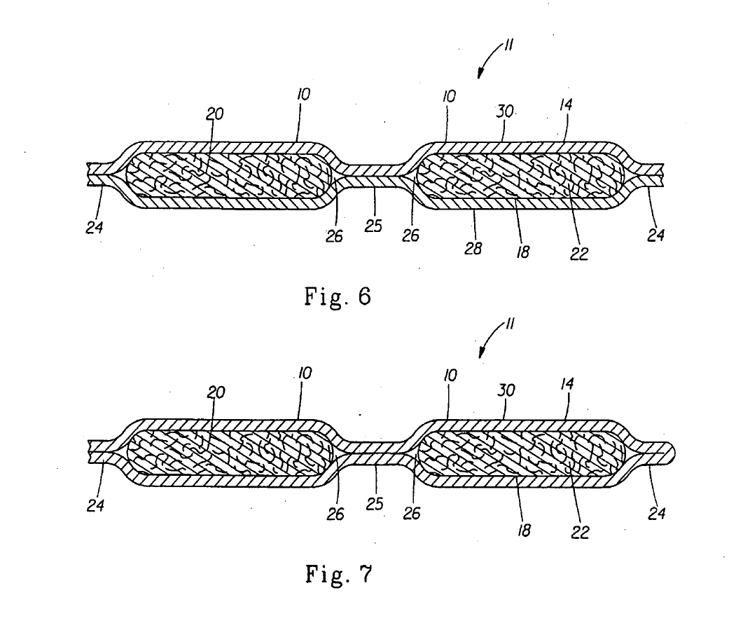

In further embodiments, as shown in FIGS. 5-9, a wipe 20 and powdered substrate 22 may be disposed in individual but joined packets 10 to form a packet combination 11. The term “packet combination”, as used herein, refers to two or more joined packets. The packet combination 11, as shown in FIG. 5, may be produced by any method known in the art, including in one embodiment, as shown in FIG. 6 (cross-section of packet combination 11 of FIG. 5 along B-B), forming both packets 10 in the packet combination 11 from a first packaging material 14 and a second packaging material 28. Wherein the first packaging material 14 and second packaging material 28 are joined about their peripheral edges 24 and along at least one dividing section 25 using means as described previously, with reference to FIGS. 1-4. The dividing section 25 is disposed between opposing peripheral edges 24 of the first packaging material 14 and second packaging material 28 to form at least two packets 10. In other embodiments as shown in FIG. 7, the packets 10 of the packet combination 11 may be formed from a first packaging material 14. The first packaging material 14 is joined about the first packaging material's 14 peripheral edges 24 and at least one dividing section 25, as described above with reference to FIG. 6.

The outer surface of the first packaging material 14 alone as shown in FIG. 7 or as shown in FIG. 6 the outer surface of the first packaging material 14 in combination with the outer surface of the second packaging material 28 form the outer surface 30 of the packets 10 and packet combination 11. The opposing surfaces of the first packaging material 14 and the second packaging material 28 form the inner surface 18, and the discrete compartments 26 where a wipe 20 or powdered substrate 22 may be disposed therein. Each packet combination 11 comprises at least one wipe 20 and one powdered substrate 22. In certain embodiments, the packet combination 11 may include a means (not shown) with the dividing section 25 that enables the packets 10 to be separated by a user, such as a line of weakness, for example perforations. In certain other embodiments, the packet combination 11 may include a means, such as a line of weakness that allows both packets 11 to be opened in one motion.

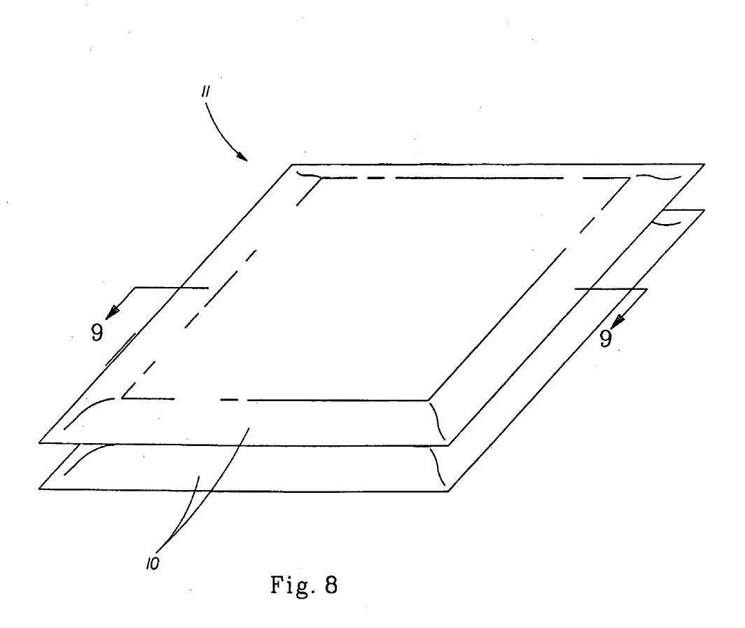

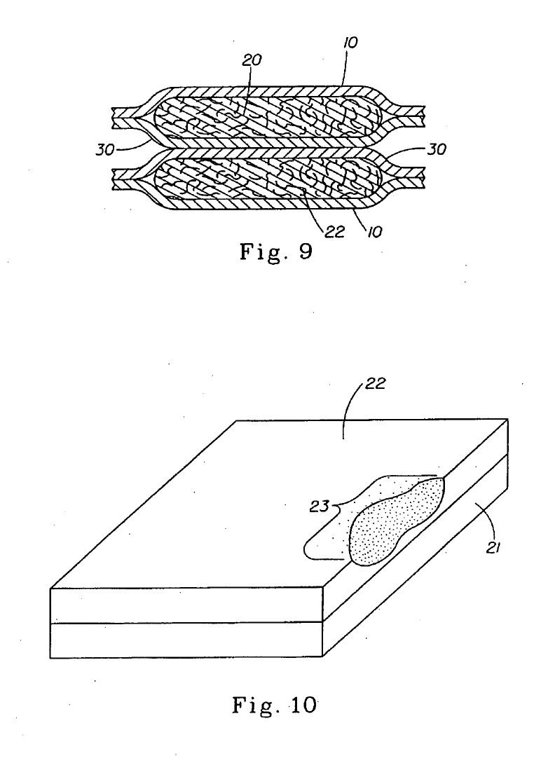

In certain other embodiments, as shown in FIGS. 8 and 9 (which is a cross-sectional view of C-C in FIG. 8), a packet combination 11 is produced when an outer surface 30 of a packet 10 may be fixedly or releasably joined to an outer surface 30 of another packet 10. In certain embodiments, as shown in FIG. 9, an individual packet 10 may contain a wipe 20 and the other packet 10 a powdered substrate 22. An adhesive can be used to join the outer surfaces 30 of the packets 10, for example, a pressure sensitive adhesive, hot melt adhesive, cold melt adhesive, or solvent based adhesives. The adhesive can be applied to an outer surface 30 of a packet 10 containing a wipe 20, and/or an outer surface 30 of a packet 10 containing the powdered substrate 22 as a continuously coated area or as a pattern, for example, dots, stripes, spirals, or beads. In certain embodiments, the packets 10 of the packet combination 11 of FIGS. 8 and 9 may be the packet 10 embodiments described above with reference to FIGS. 1-4.

Packaging materials may be made of paper, metal foil, polymer film, metalized polymer film, and/or other materials known by one of ordinary skill in the art for packaging wipes. If more than one packaging material is used, the packaging materials may be made from the same or different materials. When the wipe is a wet wipe, any material which can be sealed to maintain the wetness of the wipe can be used for the packaging material. For example, laminates with a layer of ethylene vinyl alcohol (EVOH), propylene vinyl alcohol (PVOH), or aluminum sandwiched between polymeric film layers, such as LDPE (low density polyethylene) can be used to maintain the wetness of the wipe. In certain embodiments, the packaging material is flushable and disintegrates. Such flushable packaging materials include silicone-treated polyvinyl alcohol films, or films coated with a polyvinyl alcohol, tissue coated or impregnated with polyvinyl alcohol, or other water soluble materials.

In any of the embodiments, a packet may be any desired shape. For example, the packet may be a square, rectangle, oval, circle, or any other desired shape in plan view. The packet can include a means for opening the packet in order to access a wipe and/or powdered substrate contained therein. For example, the packet can have a scored or die cut line of weakness to allow the packet to be opened easily with a tab, easy peel label, or any other opening mechanism; a “Dry-edge” sticker mechanism, such as a sticker with a dry edge for grasping; releasable adhesives; releasable heat and/or pressure seals; perforations; rupture seals; tear seals; or the packet can be made of a relatively easy to tear material such as, an easy to tear film.

The wipe may comprise a woven or nonwoven web of natural fibers, synthetic fibers, or mixtures of natural and synthetic fibers. Natural fibers may include cellulosic fibers, such as wood pulp fibers, cotton, and rayon. Synthetic fibers may include fibers such as, polyolefins, for example polyester and polypropylene fibers. In certain other embodiments, the wipe can comprise a paper material, for example, paper material that is made of a wet-laid material in a manner used to produce toilet tissue or facial tissue. In certain embodiments, the wipe can comprise silk and/or foam, such as foam disclosed in U.S. Pat. No. 5,260,345 issued Nov. 9, 1993 to DesMarais et al.; U.S. Pat. No. 5,387,207 issued Feb. 7, 1995 to Dyer et al.; U.S. Pat. No. 5,817,704 issued Oct. 6, 1998 to Shiveley et al.; U.S. Pat. No. 5,550,167 issued Aug. 27, 1996 to DesMarais; U.S. Pat. No. 5,827,909 issued Oct. 27, 1998 to DesMarais; U.S. Pat. No. 6,365,642 issued Apr. 2, 2002 to Dyer et al. The wipe may be capable of being flushed in a toilet (i.e., it is “flushable”). For example, the wipe may disintegrate into pieces sufficiently small, such that when being transported in the sewer system the pieces do not plug any element of the sewer system.

The wipe can be any size or shape that may be used for cleansing the skin, or providing other benefits (as described below) when using or changing a hygiene article. For example, in certain embodiments, the wipe may be rectangular or circular. In certain embodiments, the wipe may be about 4 square inches (about 25 square centimeters) in size to about 9 square inches (about 50 square centimeters) in size. In certain other embodiments, the wipe may be between about 16 square inches (about 100 square centimeters) in size to about 50 square inches (about 320 square centimeters) in size.

The wipe may be textured, patterned embossed, dyed, printed with ink, clear polymer or colored polymer; or combinations thereof. For example, the wipe may be printed or dyed to give a visual signal of an active ingredient. The wipe may be patterned by hydroforming or any other method known in the art.

The wipe can be a wet wipe that comprises a liquid-based solution. In certain embodiments, the wipe can comprise a nonwoven fabric impregnated with the liquid-based solution. The liquid-based solution can include, for example, surfactants, alcohols, fragrance compositions, antimicrobial agents, pH buffers, anti-itch agents, odor control agents, cooling agents, preservatives, combinations thereof, or any other substances known in the art for inclusion in wet wipes. The wipe can also contain substances such as silicones that can inhibit body exudates from adhering to the user's body. The liquid-based solution can also comprise a lotion, for example, an aqueous or non-aqueous lotion. In certain embodiments, the lotion can increase the cleaning capabilities of the wipe by aiding removal of soiling from the skin. The lotion can also provide skin benefits by reducing friction between the skin and the wipe, and/or by containing materials that can soothe the skin, for example, aloe, Vitamin E, or chamomile. Other embodiments can include dry wipes or wipes containing non-aqueous cleaning solutions such as, e.g., mineral oils, and the emollient described in U.S. Pat. No. 4,481,243 issued Nov. 6, 1984 to Allen et al.

One or more fragrance compositions can be included in the liquid-based solution in order to produce a scented wipe. Alternatively, the one or more fragrance compositions can be included in the wipe separately from any liquid-based solution, for example, by applying the fragrance composition to the wipe prior to addition of a liquid-based solution. The wipe can also include a separate component, for example, a carrier material comprising a fragrance composition.

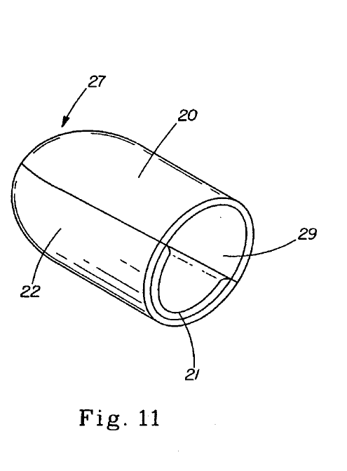

The powdered substrate 22 as shown in FIG. 10, may be any unitary substrate which releasably comprises a powder 23 suitable for application to the skin. In certain embodiments, for example the powdered substrate may be a fibrous sheet, film, foam, sponge, netting, puff or structure such as a brush, which has a powder applied thereon and/or therein. In certain embodiments a fibrous sheet may comprise a nonwoven material, wherein the non-woven material may be comprised of synthetic fibers and/or natural fibers. The powdered substrate may be a pocket formed from one or more materials, with powder contained in the space there between, wherein at least one of the materials is permeable to the powder. The powdered substrate may be a bag like structure containing powder. Further, the powdered substrate may be a powder dispensing brush such as model no. 1024, produced by ELUCI INTERNATIONAL, INC. East Rutherford, N.J. In certain embodiments, a unit dose of about 0.05 grams to about 10 grams of powder is on and/or in the powdered substrate. In other embodiments, a unit dose of about 0.1 grams to 5 grams is on and/or in the powdered substrate. In certain embodiments, a unit dose of about 0.01 grams to about 8 grams of powder is applied to the skin from the powdered substrate. In other embodiments, a unit dose of about 0.05 grams to about 3 grams of powder is applied to the skin from the powdered substrate.

In certain embodiments, the wipe may be combined with the powdered substrate in a form other than a packet, or packet combinations, as described previously with reference to FIGS. 1-9. In certain embodiments, a powder dispensing brush may have a compartment or carrying case designed to hold a wipe. Additionally, a wipe may be combined with a reusable puff, such that the puff comprises a compartment or carrying case designed to hold a wipe. Further, the wipe and powdered substrate can be packaged in a refillable hard plastic container, such as a two-sided compact.

In certain embodiments, As shown in FIG. 10 the powdered substrate 22 may have a powder impermeable layer 21, such as a barrier layer made of film, or other material impermeable to powder. Such a powder impermeable layer 21 may be positioned between the powdered surface of the powdered substrate 22 and the user's hand to prevent powder 23 from transferring to the user's hand. The powdered substrate may also comprise a handle of non-woven fibers, woven fibers, yarn, ribbon, or film material to make the powdered substrate easier to hold. The powder may be applied to the powdered substrate by spraying, dusting, coating, shaking, or any other manner known to one of ordinary skill in the art. The powder may be held on the substrate by static force, pore size of material or structure forming the powdered substrate, adhesive, or combinations thereof.

In certain embodiments, as shown in FIG. 11 the wipe 20 and powdered substrate 22 may be combined to form a two-sided mitt 27 with a central compartment 29 capable of holding one or more fingers. One side of the mitt 27 may be a wipe 20, and the other side a powdered substrate 22. The wipe 20 and powdered substrate 22 sides of the mitt 27 may have the properties disclosed previously for wipes 20 and powdered substrates 22. In certain embodiments, as described previously, the mitt 27 may have a powder impermeable layer 21 on the side of the mitt 27 having the powdered substrate 22. The powder impermeable layer 21 may be composed of a film that is between the fingers and the powdered substrate 22 side of the mitt 27 to prevent powder from transferring to the fingers.

As used herein, the term “powder” is defined as a substance comprised of ground, pulverized, or otherwise finely dispersed solid particles. In certain embodiments, the powder may have a particle size in the longest dimension of from about 1 micron to about 100 microns. In certain other embodiments, the powder may have a particle size in the longest dimension from about 5 microns to about 40 microns. The particle shape of the powder may be spherical, ellipsoidal, or irregular. The powder may contain moisture absorbers, a carrier, skin feel components, odor control agents, anti-microbial agents, antiperspirants, skin protectants, emollients, skin moisturizers, anti-oxidants, binders to help the adhesion to skin, flow aids or anti-caking aids, botanicals, colorants, fragrances, or preservatives. The powder may be white, colored, or contain colored particles to give a visual signal of the active ingredients. Examples of powders include body powders, facial powders, talcum powders, clay powders, and corn-starch powders.

Currently, there is no convenient method to apply powder to the body when outside the home. Users apply powder at home after they exit the shower and before they get dressed. There are travel size powder shakers, but they are not convenient. Using one in a public restroom would likely result in getting powder on the floor and on the user's clothing. A powdered substrate eliminates the mess associated with traditional powder application. Further, a disposable powdered substrate has the advantage of better hygiene and convenience in that it is thrown away after use.

As used herein the term “user” refers to a person who uses a wipe and/or powdered substrate on either themselves or another. The term “wearer” as used herein refers to the wearer of a hygiene article. However, the wearer and user may be the same person or they might be different. For example, a user could be the person using a wipe and/or powdered substrate to clean an infant who would be considered the wearer of a hygiene article, in this case a diaper. Further, the wearer of a sanitary napkin would also be the user of a wipe and/or powdered substrate.

A wipe may be used by a user to clean menses and/or other body exudates from the wearer's body. The cleaning of menses is particularly important because when menses leaves the wearer's body, it tends to smear over the pudendal region of the wearer's body and be retained on the skin and pubic hair. Furthermore, the menses often dries on the skin and in the pubic hair, which makes later cleansing difficult.

A wipe provides for physical cleansing, which provides increased physical comfort, as well as psychological comfort. The wipe may also be used to reduce soiling of the user's undergarment. One mechanism that causes undergarment soiling is the transfer of menses, or urine, from soiled body surfaces to the user's undergarment. In addition, the use of the wipe may also provide a reduction in the odor associated with menstruation and incontinence.

A powdered substrate may be utilized by the user to apply powder to a desired area of the body, undergarment, or hygiene article. For example, the powdered substrate can be used to apply powder directly to the pudendal region of a user before a sanitary napkin is used. Alternatively, the powdered substrate can be used to apply powder to a sanitary napkin. Upon use, the sanitary napkin will provide the powder to the pudendal region of the user. The powder may, in certain embodiments, provide to the user a comfort effect such as moisture or wetness reduction (feeling of dryness); provide fragrance immediately, or over time; a reduction in malodor; reduction in perspiration; reduction in itching; moisturizing the skin; a reduction in chafing; prevention from over drying; a cooling/soothing effect as provided, for example, by camphor; improved skin health/medicinal effect, such as the reduction in microbial growth. The powdered substrate may be used before, or after, the wipe.

One advantage of the wipe and powdered substrate is that combinations can be provided, wherein the wipe and powdered substrate confer to the user a greater benefit when used in combination, as compared to using a wipe or powdered substrate alone. For example, in certain embodiments the wipe provides an initial clean feeling to the wearer through the removal of irritants. The powder provides an on-going clean and dry feeling by continuing to absorb moisture. In addition, the powder can provide other beneficial effects to the skin by containing anti-microbial agents, emollients and/or moisturizers to maintain and restore skin health. The powder can also be used to reduce, or prevent, chafing caused by friction between the skin and hygiene article or clothing. In other embodiments, the wipe can remove perspiration, and the powder can contain an antiperspirant. The wipe can remove irritants, and the powder may contain anti-itch ingredients. The wipe can remove odor causing components, and the powder can release fragrance and/or odor control agents after application. In certain embodiments, the wipe may be used to moisten the skin and/or hair of a user. A powder is then applied to a user's skin and/or hair using a powdered substrate, which releases a fragrance upon contact with the moisture left by the wipe. Since the wipe will wet the skin and/or hair of the user, the powder will likely better adhere to the skin and/or hair, leaving less powder mess than powder being applied alone.

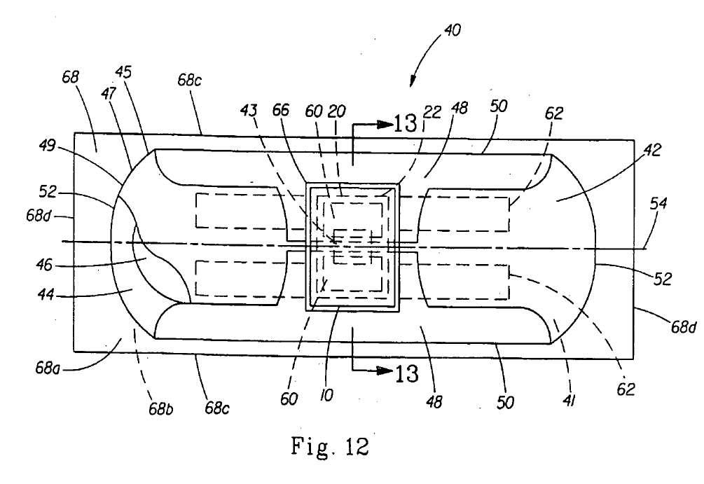

As shown in FIG. 12, in certain embodiments, the present invention comprises any of the packets 10 and/or packet combinations 11 described previously with reference to FIGS. 1-9 with a disposable hygiene article, which may be a sanitary napkin 40. The perimeter of the sanitary napkin 40 is defined by two longitudinal side margins 50, and two lateral side margins 52. The sanitary napkin 40 features a liquid pervious topsheet 42, a liquid impervious backsheet 44, an absorbent core 46, and at least one flap 48 extending from a longitudinal side margin 50 of the sanitary napkin 40. In certain embodiments, the sanitary napkin 40 comprises two symmetrically opposite flaps 48, one extending from each longitudinal side margin 50 of the sanitary napkin 40.

Associated with the sanitary napkin 40 may be an attachment means 60 and/or attachment regions 62, for releasably affixing the sanitary napkin 40 to the undergarment of a wearer. The attachment means 60 and/or attachment regions may comprise adhesives, such as pressure sensitive adhesives. However, other equivalent means may also be used. For example, adhesives which are not pressure sensitive adhesives, hook-type fasteners such as VELCRO®, selectively activated attachment materials such as those described in U.S. Pat. No. 5,662,758 issued Sep. 2, 1997 to Hamilton and U.S. Pat. No. 6,099,940 issued Aug. 8, 2000 to Hamilton et al., or combinations thereof may be used. It will be recognized by those of skill in the art, however, that these are not the only means.

Each flap 48 may also have one or more attachment means 60. In certain embodiments, the attachment means 60 is associated with the face of the flap 48, which contacts the undergarment of the wearer. That is, each flap 48 has an undergarment contacting face, or portion that contacts the wearer's undergarment, in use. As will be understood from the description herein, the undergarment contacting face of the flap 48 is the portion that contacts the outside surface of the wearer's undergarment when the flap 48 is in an in-use folded position. Also, the sanitary napkin 40 may have an attachment region 62 associated with the area of the central portion of the sanitary napkin 40 (as defined by the longitudinal side margins 50), which contacts the undergarment of the wearer. The attachment region 62 may be located on the outwardly oriented face 41 (face which contacts the undergarment of the wearer) of the backsheet 44.

The sanitary napkin 40 has a longitudinal centerline 54. As used herein, the term “longitudinal” refers to an imaginary line, axis, or direction, of the sanitary napkin 40, which line, axis, or direction, is typically parallel to the longitudinal side margins 50 of the sanitary napkin 40. The term “lateral”, as used herein, refers to an imaginary line, axis, or direction, generally orthogonal the longitudinal direction within the plane of the sanitary napkin 40.

With continuing reference to FIG. 12, the topsheet 42 of the sanitary napkin 40 is oriented towards and contacts the body of the wearer to receive bodily discharges. In certain embodiments, the topsheet 42 is liquid pervious, and may be flexible and nonirritating to the skin. As used herein, the term “flexible” refers to materials which are compliant and readily conform to the shape of the body, or respond by easily deforming in the presence of external forces. Topsheets 42 may be made from nonwoven materials or perforated polyolefinic films. In certain embodiments, the topsheet 42 has a plurality of apertures to permit liquids deposited thereon to pass through to the absorbent core 46. Such apertures may, but need not, be present in the flaps 48. Topsheets 42 may be made in accordance with U.S. Pat. No. 4,342,314 issued Aug. 3, 1982 to Radel et al. and U.S. Pat. No. 4,463,045 issued Jul. 31, 1984 to Ahr et al.

The backsheet 44 may be any flexible, liquid resistant, and in certain embodiments, liquid impervious material, for example, a polyolefinic film, such as a polyethylene film. The backsheet 44 prevents discharges collected by and contained in the sanitary napkin 40, and particularly discharges absorbed by the absorbent core 46, from escaping the sanitary napkin 40 and soiling the clothing and bedding of the wearer. In certain embodiments, the backsheet 44 may also be impervious to malodorous gases generated by absorbed bodily discharges, so that the malodors do not escape and become noticed by the wearer. In certain other embodiments, the backsheet 44 may also permit gases or vapors to pass through it (i.e. be breathable), while still resisting the passage of liquids there through. Further, the backsheet 44 may be made of a soft cloth-like material, which is hydrophobic relative to the topsheet 42, for example, a polyester or polyolefinic fiber backsheet 44. In certain embodiments, a soft cloth-like backsheet 44 material may be a laminate of a polyester non-woven material lamina, and a film such as described in U.S. Pat. No. 4,476,180 issued Oct. 9, 1984 to Wnuk.

The topsheet 42 and the backsheet 44 may be joined along their peripheries 45, 47 using known techniques, either entirely so that the entire perimeter 49 of the sanitary napkin 40 is circumscribed by such joinder, or are partially peripherally joined at the perimeter 49 of the sanitary napkin 40. Such a sanitary napkin 40 has two mutually opposed major faces, one defined by the topsheet 42, (body faceable side) and one defined by the backsheet 44 (undergarment faceable side).

As stated previously, the outwardly oriented face 41 of the backsheet 44 may comprise one or more attachment regions 62 for attaching the sanitary napkin 40 to the undergarment of the wearer. In certain embodiments, the attachment region 62 may be pressure sensitive adhesive. The attachment region 62 may be continuous or intermittent. In certain embodiments, the backsheet 44 may have the attachment region 62 oriented in two longitudinal strips, as shown in FIG. 12, with one attachment region 62 on each side of the longitudinal centerline 54.

The absorbent core 46 collects and contains bodily discharges, particularly menses, which traverses through the liquid permeable topsheet 42. The absorbent core 46 is the component of the sanitary napkin 40, which receives and retains the bodily discharges. Like the topsheet 42 and backsheet 44, the absorbent core 46 may be conformable and nonirritating to the skin. In certain embodiments, the absorbent core 46 may be rectangular or hourglass shaped. In certain embodiments, the absorbent core 46 has two opposed faces, one oriented towards the backsheet 44, and one oriented towards the topsheet 42.

The absorbent core 46 may comprise absorbent material. The absorbent material may comprise airfelt, such as cellulose wadding and fibrated communition pulp; tissue paper; absorbent gelling materials, foam, or combinations thereof. If the absorbent core 46 comprises tissue paper, tissue paper made in accordance with U.S. Pat. No. 4,191,609 issued Mar. 4, 1980 to Trokhan may be used. Likewise, if the absorbent core 46 comprises absorbent gelling materials, absorbent gelling materials made in accordance with U.S. Pat. Re. No. 32,649 issued Apr. 19, 1988 to Brandt et al. may be used. Further, if the absorbent core 46 comprises foam, foam disclosed in U.S. Pat. No. 5,260,345 issued Nov. 9, 1993 to DesMarais and U.S. Pat. No. 5,387,207 issued Feb. 7, 1995 to Dyer et al. may be used.

In certain embodiments, the absorbent core 46 is interposed between the topsheet 42 and backsheet 44. This prevents the absorbent material of the absorbent core 46 from shredding or becoming detached while the sanitary napkin 40 is worn, and to ensure proper containment of bodily discharges. The absorbent core 46 may be joined to the topsheet 42 and backsheet 44. Joining may be by bonding the absorbent core 46 to the topsheet 42, or the backsheet 44, using an adhesive. Such adhesive (not shown) may be applied in a spray pattern, such as a spiral or longitudinally oriented beads.

The sanitary napkin 40 may also comprise a flap 48 extending from a longitudinal side margin 50 of the sanitary napkin 40, and in certain embodiments, one flap 48 extends from each longitudinal side margin 50 of the sanitary napkin 40. The flaps 48 may be of any shape desired, which facilitates the attachment of the sanitary napkin 40 to the undergarment of a wearer. The flaps 48 may be comprised of an integral and contiguous extension of the topsheet 42, the backsheet 44, or a laminate of both. Alternatively, the flaps 48 may be made of a separate and independent piece of material joined to the longitudinal side margin 50 of the sanitary napkin 40. Each flap 48 has one face generally coextensive of the topsheet 42, and a mutually opposed face generally coextensive of the backsheet 44.

The flaps 48 may have an attachment means 60 for attaching one face of a flap 48 to the wearer's undergarment, or to another other flap 48. In certain embodiments, if the attachment means 60 should be disposed on the face of the flap 48 generally coextensive of the backsheet 44, so that when the flaps 48 are wrapped around the crotch portion of the wearer's undergarment, the attachment means 60 will contact the outside of the wearer's undergarment. Folding the flaps 48 in the configuration, as shown in FIG. 12, exposes the attachment means 60 on the face of the flaps 48.

To prevent contamination and blocking of the attachment means 60, the attachment means 60 of each flap 48 may be covered with a release strip 66. A release strip 66 may cover each attachment means 60 individually, or as in the embodiment shown in FIG. 12, a single release strip 66 may cover the attachment means 60 of each flap 48. The release strips 66 described herein may be made of kraft paper, calendared paper, or any other materials well known in the art for such purpose. The face of the release strip 66, which contacts the attachment means 60, may have a release coating, such as silicone, to facilitate the removal of the release strip 66 from the flaps 48.

The sanitary napkin 40 of the present invention, having a packet 10 or packet combination 11 associated therewith, may optionally be provided as an individually wrapped sanitary napkin 40. Such sanitary napkins are described in U.S. Pat. No. 4,462,166 issued Jul. 31, 1984 to Furlong. As shown in FIG. 12, the sanitary napkin 40 may be superimposed on a releasable wrapper (or “release paper wrap”) 68. (The releasable wrapper need not be paper, however.) The releasable wrapper 68 underlays and is releasably joined to the outwardly oriented face 41 of the backsheet 44. In the embodiment shown, the releasable wrapper 68 is slightly larger than the sanitary napkin 40 as it is defined by the longitudinal 50 and lateral 52 side margins. The releasable wrapper 68 contacts the attachment region 62 of the backsheet 44.

The releasable wrapper 68 has a perimeter defined by longitudinal edges 68c and lateral edges 68d. The releasable wrapper 68 has opposed faces. One face is an inwardly oriented face 68a, which is oriented towards the backsheet 44. The other is the outwardly oriented face 68b of the releasable wrapper 68, which is opposed to the inwardly oriented face 68a and oriented away from the sanitary napkin 40.

In certain embodiments, the inwardly oriented face 68a is release coated to facilitate easy and convenient manipulation of the releasable wrapper 68, and particularly, separation from the attachment region 62. For example, silicone release coatings may be used. The releasable wrapper 68 may be zone coated with the release coating only in the areas of the attachment region 62, or may be entirely release coated throughout the inwardly oriented face 68a as desired. The releasable wrapper 68 may be made of films, kraft paper, calendared paper, or other materials, as are well known in the art. The releasable wrapper 68 may be made of one or more sheets of material.

As stated previously, the sanitary napkin 40 of the present invention has at least one packet 10, or packet combination 11, containing a wipe 20 and powdered substrate 22. When describing the various embodiments of the present invention comprising a packet 10 and/or a packet combination 11 with a sanitary napkin 40, the term packet 10 shall be understood to include not only at least one packet 10, but also a packet combination 11, or any other previously described embodiments which contain a wipe and powdered substrate. The packet 10 may be located anywhere on the sanitary napkin 40 (including the releasable wrapper 68) where a wearer may access such packet 10. In certain embodiments, the packet 10 can be either releasably or fixedly joined to the sanitary napkin 40. A joining means 43 may be used for joining the packet 10 to the sanitary napkin 40. Any joining means 43 known by one in the art for joining a packet as described herein to a sanitary napkin may be used, for example, adhesive or hook type fasteners such as VELCRO may be used.

In certain embodiments, as shown in FIG. 12, the packet 10 may be associated with the release strip 66 of the sanitary napkin 40. In this embodiment, the packet 10 is joined to the release strip 66 by a joining means 43 such as adhesive.

The convenient inclusion of a packet 10 with an individual sanitary napkin 40 in the manner described herein provides several advantages. The packet 10 of the present invention is provided in such a way as to minimize the amount of extra material required to produce the sanitary napkin 40/packet 10 combination. A portion of the packet 10, as more fully described below, can serve as the release strip 66. Also, due to the placement of packet 10 there is little difference in the way the user installs the sanitary napkin 40, whether or not the user actually uses the packet 10. In fact, the user may choose not to use the packet 10, and does nothing different in otherwise using the sanitary napkin 40. That is, as shown below, the packet 10, can be simply removed as the release strip 66 would otherwise be removed.

The packet 10 is packaged in a manner that is convenient for the user to use during the installation of a sanitary napkin 40 in their undergarment. That is, to access the packet 10 of the present invention requires little more effort than is already being expended by the user. This is an advantage as the user is often concerned with speed, ease of use, and discreteness during the changing of a sanitary napkin 40. For example, the user may be in a location, such as a public restroom stall, that makes movement difficult, particularly while trying to maintain cleanliness. At these times, the user does not want to have to manage complicated or inconvenient steps to access an item such as the packet 10 while at the same time installing a sanitary napkin 40.

Therefore, to use a sanitary napkin 40 of the present invention, the user typically removes the release strip 66 and attach the attachment region 62 to the crotch of their undergarment. The user would then remove the packet 10 with, or without, also removing the release liner 66. Depending on the particular embodiment, as described herein, the release liner 66 may, or may not, be an element wholly separate from the packet 10. The user then folds the flaps 48 around the side edges of the crotch portion of their undergarment, and, if attachment means 60 are present, attaches flaps 48 to the underside of their undergarment. The user then, if desired, uses the wipe 20 and powdered substrate 22 provided by opening the packet 10, using the wipe 20 and powdered substrate 22, and disposing of the wipe 20, powdered substrate 22, and packet 10 appropriately.

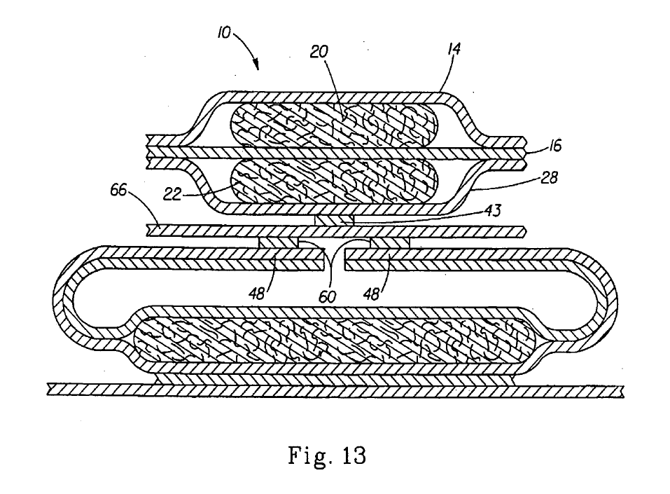

The drawing figures show several nonlimiting alternative ways of associating a packet 10 with a sanitary napkin 40. One embodiment of the present invention is shown in FIG. 13, which is a cross-section (D-D) of the sanitary napkin of FIG. 12. In this embodiment, the wipe 20 and powdered substrate 22 are packaged in a packet 10. As described previously, above with reference to FIG. 3, the packet 10 is comprised of a first packaging material 14, a second packaging material 28, which form the outer surface 30, and a third packaging material 16. The packet 10 is joined to the release strip 66 by any joining means 43 known in the art.

In the embodiment disclosed in FIG. 12, it is not necessary that the packet 10 be easily removable from the release strip 66. In use, the user would remove the packet 10, which could then beneficially affect removal of the release strip 66. The user could then, if desired, open the packet 10 with the release strip 66 still joined to the packet 10. The user could then simply dispose of the packet 10 and release strip 66 as a unit. However, in another embodiment, the joining means 43, between the packet 10 and release strip 66 can have less peel strength than that of the attachment means 60 between release strip 66 and the backflaps 48. This allows the user to easily remove the packet 10, use the enclosed wipe 20 and powdered substrate 22, and then remove the release strip 66.

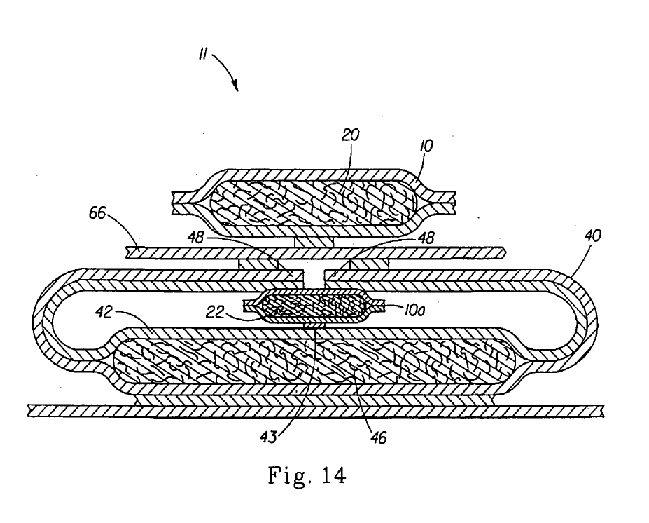

In another embodiment, as shown in FIG. 14, which is also a cross-section of D-D, a packet combination 11 having a packet 10 containing a wipe 20 or powdered substrate 22, which for the purposes of illustration only, is shown in FIG. 14 as a packet 10 containing a wipe 20, is joined to the release strip 66 as described above with reference to FIG. 12. However, in certain other embodiments, a packet 10 could be joined to flaps 48 of the sanitary napkin 40 as described below. In addition, the other packet 10a of the packet combination 11 containing a powdered substrate 22 may be disposed on the topsheet 42 of the sanitary napkin 40. In certain embodiments, as shown in FIG. 14, the packet 10a may optionally be disposed on the topsheet 42 between the absorbent core 46 and the flaps 48. In this embodiment, the flaps 48 serve to completely or partially enclose the packet 10a. The packet 10a may be joined to the topsheet 42 using a joining means 43. Alternatively, the packet 10a may be joined to the topsheet 42 by being held in place by the packet's 10a placement on the topsheet 42. For example, in an embodiment where the packet 10a is disposed between the absorbent core 46 and the flaps 48, the pressure exerted by the flaps 48 and absorbent core 46 on the packet 10a is sufficient to hold the packet 10a in place. In still further embodiments, where the packet 10a is disposed on the topsheet 42, the folding of the sanitary napkin 40 (and in some embodiments the releasable wrapper 68) as described later, serves to join the packet 10a to the sanitary napkin 40, by holding the packet 10a in place.

An order of use can be dictated by locating the packet 10 containing the wipe 20 and the packet 10a containing the powdered substrate 22 in specific positions on the sanitary napkin 40. For example, in certain embodiments, packets 10, 10a could be arranged in a manner such that upon using the sanitary napkin 40, as described previously, the user is exposed to one packet 10 before the other 10a, as shown in FIG. 14. This would allow a user opening and using the sanitary napkin 40 the option of opening a single packet 10, 10a and possibly using the contents and disposing of said packet 10, 10a, before having to open another packet 10, 10a. For example, once again with reference to FIG. 14, the packet 10 joined to the release strip 66 could contain a wipe 20, which allows the user to first clean any residual menses from the pudendal region. The user could then dispose of the wipe 20 and packet 10 before continuing to use the sanitary napkin 40. When the user desires, they then may access the other packet 10a containing the powdered substrate 22 so as to apply powder to themselves, the sanitary napkin 40 or any other desirable area. In certain embodiments, packets 10, 10a may have indicia on them, such as printing to communicate to a user a sequence in which the wipe 20 and powdered substrate 22 may be used. The indicia can take many forms, for example, the packet 10, 10a could be printed with numbers, written directions, pictures or diagrams, to visually indicate to the user the sequence of use, or alternatively different sizes and shapes of the packets 10, 10a could be used to indicate the proper order of use.

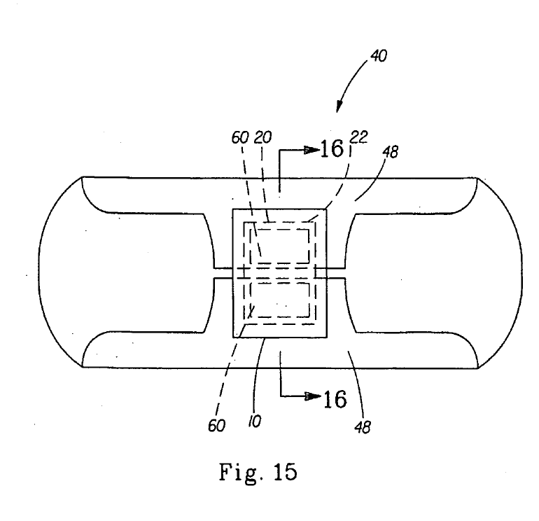

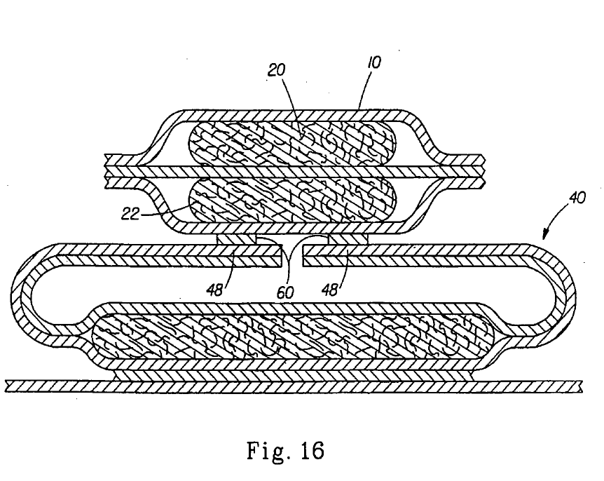

In certain embodiments, as shown in FIGS. 15 and 16 (cross-section E-E of the sanitary napkin 40 of FIG. 15), the packet 10 containing both a wipe 20 and powdered substrate 22 may also function as the release strip. The packet 10 can be joined to the flaps 48, for example, by using an attachment means 60. Where applicable the package material of the packet 10 functioning as the release strip may be joined to the flaps 48 before the packet 10 is completely formed, i.e., any time before the packet 10 is sealed. The material of the packet 10 functioning as the release strip may be made from the same material as the release strip 66 described previously. However, the packet 10 can also be made in a separate operation, and subsequently attached by known methods for joining a release strip to a sanitary napkin 40.

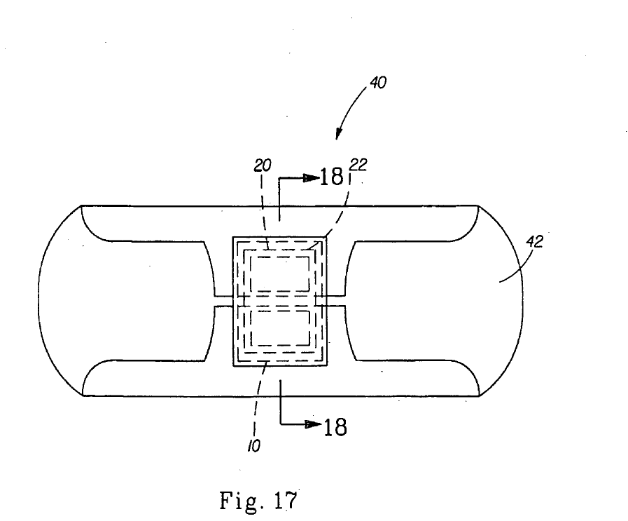

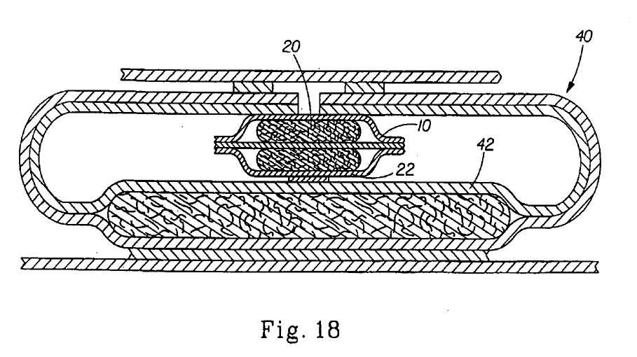

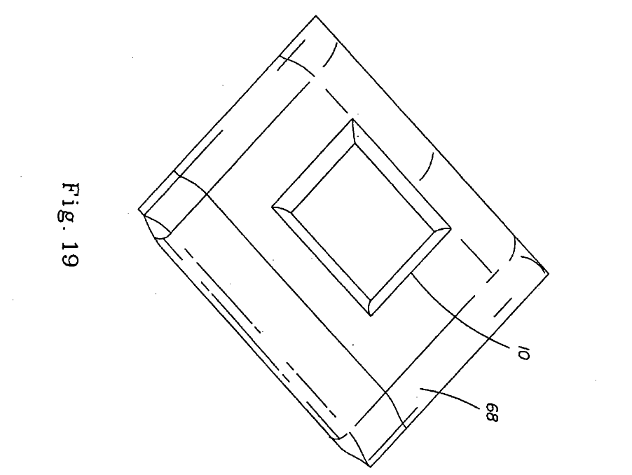

In certain embodiments as shown in FIGS. 17 and 18, (cross-section F-F of the sanitary napkin 40 of FIG. 17) a packet 10 containing both a wipe 20 and a powdered substrate 22 may be disposed on the topsheet 42 of the sanitary napkin 40, as described above with reference to the packet 10a of FIG. 14

As shown in FIG. 19, a packet 10 may be joined to the releasable wrapper 68 of a sanitary napkin. The sanitary napkin and releasable wrapper 68 may be folded into a final configuration by any means known in the art such as the method disclosed in U.S. Pat. Nos. 5,569,230 issued Oct. 29, 1996 to Fisher et al. and 6,911,022 issued Jun. 28, 2005 to Steger et al. The packet 10 may be joined anywhere on the releasable wrapper 68 which includes the inwardly oriented face 68a and outwardly oriented face 68b as shown in FIG. 12. The packet 10 may be joined to the releasable wrapper 68 using any means known in the art, such as adhesive. In certain embodiments, there may be a packet 10 joined to the releasable wrapper 68 and at least one packet 10 joined to the sanitary napkin, as described above with reference to FIGS. 12-19.

Although FIGS. 12-19 show a sanitary napkin 40 as the hygiene article, other hygiene articles may be used, such as, tampons, pessaries, inter labial pads, adult incontinence products, training pants, and baby diapers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

It will be understood that the embodiment(s) described herein is/are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

1. A hygiene article comprising:
a.) a hygiene article; and
b.) a packet combination joined to the hygiene article, wherein the package combination comprises;
i.) a first packet comprising a first packaging material, the first packaging material defining an outer surface of the first packet and an internal space, wherein the internal space is a first discrete compartment;
ii.) a second packet comprising a second packaging material, the second packaging material defining an outer surface of the second packet and an internal space, wherein the internal space is a second discrete compartment;
iii.) wherein at least one wipe is disposed within the first discrete compartment;
iv.) wherein at least one powdered substrate is disposed within the second discrete compartment; and
v.) wherein the first packet and the second packet are joined.

2. The hygiene article of claim 1 wherein the hygiene article comprises a pair of flaps, wherein at least one of the first packet or the second packet of the packet combination is joined to the flaps.

3. The hygiene article of claim 1 wherein the hygiene article comprises a topsheet, wherein at least one of the first packet or the second packet of the packet combination is joined to the topsheet.

4. The hygiene article of claim 1 wherein the hygiene article comprises a backsheet, wherein at least one of the first packet or the second packet of the packet combination is joined to the backsheet.

5. The hygiene article of claim 1 wherein the hygiene article comprises a releasable wrapper, wherein at least one of the first packet or the second packet of the packet combination is joined to the releasable wrapper.

6. The hygiene article of claim 1 wherein the hygiene article comprises a releasable wrapper and a pair of flaps, wherein the first packet is joined to the releasable wrapper and the second packet is joined to the pair of flaps.

7. The hygiene article of claim 1 wherein the hygiene article comprises a topsheet and a pair of flaps, wherein the first packet is joined to the pair of flaps and the second packet is joined to the topsheet.

8. The hygiene article of claim 1 wherein the wipe is a wet wipe.

9. The hygiene article of claim 1 wherein the powdered substrate is at least one of a fibrous sheet, film, foam, sponge, netting, puff, brush, or bag.

10. The hygiene article of claim 1 wherein the hygiene article is a sanitary napkin.

11. The hygiene article of claim 1 including at least one additional packet.

12. A hygiene article comprising:
a.) a hygiene article; and
b.) a packet joined to the hygiene article, wherein the packet comprises;
i.) packaging material, the packaging material defining an outer surface of the packet and an internal space;
ii.) an internal divider, the internal divider disposed in the internal space and joined to the packaging material to define a first discrete compartment and a second discrete compartment;
iii.) wherein at least one wipe is disposed within the first discrete compartment; and
iv.) wherein at least one powdered substrate is disposed within the second discrete compartment.

13. The hygiene article of claim 12 wherein the hygiene article comprises a pair of flaps, wherein the packet is joined to the flaps.

14. The hygiene article of claim 12 wherein the hygiene article comprises a topsheet, wherein the packet is joined to the topsheet.

15. The hygiene article of claim 12 wherein the hygiene article comprises a backsheet, wherein the packet is joined to the backsheet.

16. The hygiene article of claim 12 wherein the hygiene article comprises a releasable wrapper, wherein the packet is joined to the releasable wrapper.

17. The hygiene article of claim 12 wherein the wipe is a wet wipe.

18. The hygiene article of claim 12 wherein the powdered substrate is at least one of a fibrous sheet, film, foam, sponge, netting, puff, brush, or bag.

19. The hygiene article of claim 12 wherein the hygiene article is a sanitary napkin.
